(19) United States
(12) Patent Application Publication
(10) Pub. No.: US 2026/0346015 A1
(43) Pub. Date: Nov. 5, 2026

(54) CONVERTING BETWEEN BIPOLAR AND UNIPOLAR ELECTRICAL STIMULATION THERAPY

(71) Applicant: MEDTRONIC, INC., Minneapolis, MN (US)

(72) Inventors: Nathan A. Torgerson, Andover, MN (US); Christian H. de Laszlo, Little Silver, NJ (US); Jerel K. Mueller, Saint Paul, MN (US); Gregg Beideman, Langhorne, PA (US); Timothy R. Abraham, Lino Lakes, MN (US); Thomas L. Chouinard, Maple Grove, MN (US)

(21) Appl. No.: 19/099,350

(22) PCT Filed: Jul. 26, 2023
(86) PCT No.: PCT/US2023/028634

§ 371 Date: Jan. 28, 2025

Related U.S. Application Data
(60) Provisional application No. 63/369,737, filed on Jul. 28, 2022.

(51) Int. Cl. A61N 1/36; A61N 1/05; A61N 1/372

(52) U.S. Cl. CPC A61N 1/36139; A61N 1/0534; A61N 1/3615; A61N 1/36171; A61N 1/36175; A61N 1/36185; A61N 1/37247

(57) ABSTRACT

Devices, systems, and techniques are described for switching between electrical stimulation having a bipolar electrode combination and electrical stimulation having a unipolar electrode combination. In one example, processing circuitry is configured to receive a first stimulation parameter set comprising a bipolar electrode combination that defines a first electrical stimulation, estimate a volume of neural activation (VNA) corresponding to the first stimulation parameter set, determine, based on the first VNA, a second stimulation parameter set comprising a unipolar electrode combination that defines a second electrical stimulation, and control an implantable medical device to deliver the second electrical stimulation instead of the first electrical stimulation.

This application is a PCT application that claims priority to and the benefit of U.S. Provisional Patent Application No. 63/369,737, filed Jul. 28, 2022, the entire contents of which is incorporated herein by reference.

### TECHNICAL FIELD

The disclosure relates to medical devices and, more particularly, to medical devices that deliver electrical stimulation therapy.

### BACKGROUND

Medical devices may be used to treat a variety of medical conditions. Medical electrical stimulation devices, for example, may deliver electrical stimulation therapy to a patient via electrodes. Electrical stimulation therapy may include stimulation of nerve, muscle, or brain tissue, or other tissue within a patient. An electrical stimulation device may be fully implanted within the patient. For example, an electrical stimulation device may include an implantable electrical stimulation generator and one or more implantable leads carrying electrodes. The electrical stimulation device may comprise a leadless stimulator. In some cases, implantable electrodes may be coupled to an external electrical stimulation generator via one or more percutaneous leads or fully implanted leads.

Medical electrical stimulators may be used to deliver electrical stimulation therapy to patients to relieve a variety of symptoms or conditions such as chronic pain, tremor, Parkinson's disease, depression, epilepsy, urinary or fecal incontinence, pelvic pain, sexual dysfunction, obesity, or gastroparesis. An electrical stimulator may be configured to deliver electrical stimulation therapy via leads that include electrodes proximate to the spinal cord, pelvic nerves, gastrointestinal organs, peripheral nerves, or within the brain of a patient. Stimulation proximate the spinal cord and within the brain are often referred to as spinal cord stimulation (SCS) and deep brain stimulation (DBS), respectively.

### SUMMARY

In general, the disclosure describes devices, systems, and techniques for converting electrode combinations from a bipolar electrode combination to a unipolar electrode combination. Electrical stimulation may be delivered in a bipolar electrode configuration which includes current delivered between two or more electrodes that are in close proximity to each other, such as electrodes disposed on the same lead. This bipolar electrode configuration may result in a volume of neural activation (VNA) for the patient that may be configured to reduce one or more symptoms associated with one or more disorders of the patient, such as tremor or Parkinson's disease in the example of DBS.

However, a physician may desire to deliver electrical stimulation that has a similar VNA using a unipolar electrode configuration. Electrical stimulation delivered using a unipolar electrode combination includes current delivered from one or more cathodes to one or more anodes remote from, or farther away from, the cathodes. For example, the one or more cathodes may be located on a lead while an anode is located on a housing of the implantable medical device (IMD) coupled to the lead.

The system described herein may be configured to convert the bipolar electrode combination to a unipolar electrode combination while maintaining a similar VNA. For example, the system may compare the VNA from the bipolar electrode combination to VNAs corresponding to respective unipolar electrode combinations in order to identify an appropriate unipolar electrode combination. In some examples, the system may convert to the unipolar electrode combination by switching all electrodes of the bipolar electrode combination to cathodes and then adding a remote anode. The system may then adjust current amplitude of one or more of the cathodes in order to approximate the VNA of the bipolar electrode combination. The system may include a user interface that has a convert button that, when selected, causes the system to determine the unipolar electrode combination and switch from the bipolar electrode combination to the unipolar electrode combination.

In one example, this disclosure describes a system including a memory and processing circuitry operably coupled to the memory and configured to: receive a first stimulation parameter set comprising a bipolar electrode combination that defines a first electrical stimulation; estimate a volume of neural activation (VNA) corresponding to the first stimulation parameter set; determine, based on the first VNA, a second stimulation parameter set comprising a unipolar electrode combination that defines a second electrical stimulation; and control an implantable medical device to deliver the second electrical stimulation instead of the first electrical stimulation.

In another example, this disclosure describes a method including receiving, by processing circuitry, a first stimulation parameter set comprising a bipolar electrode combination that defines a first electrical stimulation; estimating, by the processing circuitry, a volume of neural activation (VNA) corresponding to the first stimulation parameter set; determining, by the processing circuitry and based on the first VNA, a second stimulation parameter set comprising a unipolar electrode combination that defines a second electrical stimulation; and controlling, by the processing circuitry, an implantable medical device to deliver the second electrical stimulation instead of the first electrical stimulation.

In another example, this disclosure describes a system including a memory comprising a first stimulation parameter set that defines a first electrical stimulation; and processing circuitry operably coupled to the memory and configured to: control a user interface to present a representation of first electrical stimulation defined by a first stimulation parameter set comprising a bipolar electrode combination; controlling, by the processing circuitry, the user interface to present a selectable icon associated with converting the first electrical stimulation defined by the first stimulation parameter set comprising the bipolar electrode combination to a second electrical stimulation defined by a second stimulation parameter set comprising a unipolar electrode combination; receiving, by the processing circuitry, user selection of the selectable icon; and responsive to receiving the user selection of the selectable icon, converting the first electrical stimulation to the second electrical stimulation defined by the second stimulation parameter set comprising the unipolar electrode combination.

In another example, this disclosure describes a method including controlling, by processing circuitry, a user interface to present a representation of first electrical stimulation defined by a first stimulation parameter set comprising a bipolar electrode combination; controlling, by the processing circuitry, the user interface to present a selectable icon associated with converting the first electrical stimulation defined by the first stimulation parameter set comprising the bipolar electrode combination to a second electrical stimulation defined by a second stimulation parameter set comprising a unipolar electrode combination; receiving, by the processing circuitry, user selection of the selectable icon; and responsive to receiving the user selection of the selectable icon, converting the first electrical stimulation to the second electrical stimulation defined by the second stimulation parameter set comprising the unipolar electrode combination.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example therapy system that includes an electrical stimulator coupled to a stimulation lead, in accordance with various techniques of this disclosure.

FIG. 2 is a block diagram illustrating the example programmer of FIG. 1 in further detail.

FIG. 3 is a block diagram illustrating the example electrical stimulator of FIG. 1 in further detail.

FIG. 4 is a block diagram illustrating an example of the electrical stimulation generation circuitry of the electrical stimulator of FIG. 3 in further detail.

FIG. 5 is a flowchart illustrating an example operation for switching from bipolar stimulation to unipolar stimulation.

FIG. 6 is a flowchart illustrating an example operation for converting bipolar stimulation to unipolar stimulation in response to user input.

FIGS. 7A and 7B are conceptual diagrams illustrating screens of an example user interface in accordance with the techniques of the disclosure.

Like reference characters refer to like elements throughout the figures and description.

### DETAILED DESCRIPTION

Devices, systems, and techniques are described herein for changing electrical stimulation from bipolar electrode combinations to unipolar electrode combinations. When programming Deep Brain Stimulation (DBS) therapy, a clinician can specify an electrode configuration and then define delivery of electrical stimulation via the electrode configuration by assigning values of one or more therapy parameters to individual electrodes and/or adjusting values of these therapy parameters. A clinician typically selects one or more electrodes to be used as cathodes and one or more electrodes to be used as anodes which specify the path for current during stimulation. This electrode combination is selected from electrodes available one or more leads adjacent the target tissue location, and may be referred to as bipolar electrodes combinations. Bipolar electrode combinations are used generally to describe anodes and cathodes located on one or more leads adjacent a target tissue, which may be referred to as multipolar electrode combinations in some examples. The clinician may also identify other parameter values that define stimulation, such as voltage or current amplitudes for each electrodes of the bipolar electrode combination, pulse frequencies, pulse widths, etc.

Bipolar electrode combinations enable stimulation to be delivered between one or more cathode and one or more anode that are relatively close to each other and near the target tissue. The resulting stimulation may be relatively precise from a spatial point of view because the electrical current, and electrical field, does not travel very far through tissue. This spatial control may be beneficial for certain anatomical locations, such as in the brain, where therapy efficacy may depend on affecting some neural networks and avoiding other neural networks. However, bipolar electrode combinations may be less energy efficient due to the proximity of the anodes to the cathodes. Since anodes and cathodes may be very close, such as within a few millimeters from each other, some electrical current may effectively travel directly between the anodes and cathodes instead of affecting tissue. Therefore, some energy does not reach the target tissue which results in higher current or voltage amplitudes in order to reach the target tissue.

As described herein, a system may be configured to convert a bipolar electrode configuration for electrical stimulation to a unipolar electrode combination. In some examples, the system may utilize volume of activation (VNA) modeling to determine an appropriate unipolar electrode combination and other parameter values for switching from the bipolar electrode configuration. The electrical stimulation generated using a bipolar electrode configuration may result in a volume of neural activation (VNA) for the patient that may be configured to reduce one or more symptoms associated with one or more disorders of the patient, such as tremor or Parkinson's disease in the example of DBS. The system can model, or estimate, this VNA based on the cathodes and anodes of the bipolar electrode combination and other parameter values, such as current amplitude. The system can then determine the unipolar electrode combination and other parameter values that will produce a VNA similar to the VNA of the bipolar electrode combination. Since the VNAs may not exactly match, the system may weight one or more characteristics of the VNA matching processing greater than other characteristics.

In addition, or alternatively, the system may convert the bipolar electrode combination to the unipolar electrode combination by switching all electrodes of the bipolar electrode combination to cathodes and then adding a remote anode. In this manner, all cathodes of the bipolar electrode combination would remain cathodes, and all anodes of the bipolar electrode combination would be switched to cathodes. The remote anode may be one or more anodes on another lead remote from the target tissue or attached to or a part of the housing the IMD coupled to the lead. In this manner, the remote electrode(s) may sink current from the cathodes. The system may then adjust current amplitude of one or more of the cathodes in order to approximate the VNA of the bipolar electrode combination.

In some examples, the physician may desire to deliver electrical stimulation that has a similar VNA using a unipolar electrode configuration. The system may include a user interface, such as a user interface presented on a clinician external programmer, that has a convert button that, when selected, causes the system to determine the unipolar electrode combination and switch from the bipolar electrode combination to the unipolar electrode combination. In other examples, the system may automatically suggest switching to a unipolar electrode combination to conserve power. The system may present a notification requesting that the user confirm the switch from the bipolar electrode combination to the unipolar electrode combination.

Electrical stimulation delivered using a unipolar electrode combination may provide similar therapeutic efficacy while reducing power consumption. This reduced power consumption may be due to one or more factors, such as the anode being unregulated for unipolar electrode configurations where no headroom is required and there is less voltage drop across circuitry. In addition, or alternatively, the anode can be a larger surface area on the housing of the IMD or another larger electrode(s) which reduces resistance to current flow from the cathodes. In addition, or alternatively, passing current over larger distances from the cathodes to the remote anode can avoid shunting (direct electrical current flow) that may occur between cathodes and anodes that are positioned close together instead of causing tissue activation. Further, anodic voltages from anodes in a bipolar electrode configuration may be less efficient to activate neurons than cathodes. For one or all of these reasons, the system may realize benefits by delivering electrical stimulation using unipolar electrode combinations. Moreover, since the clinician may be comfortable programming therapy using bipolar electrode combinations, the clinician can continue to identify effective bipolar electrode combinations and then convert those combinations to unipolar electrode combinations that may be more energy efficient.

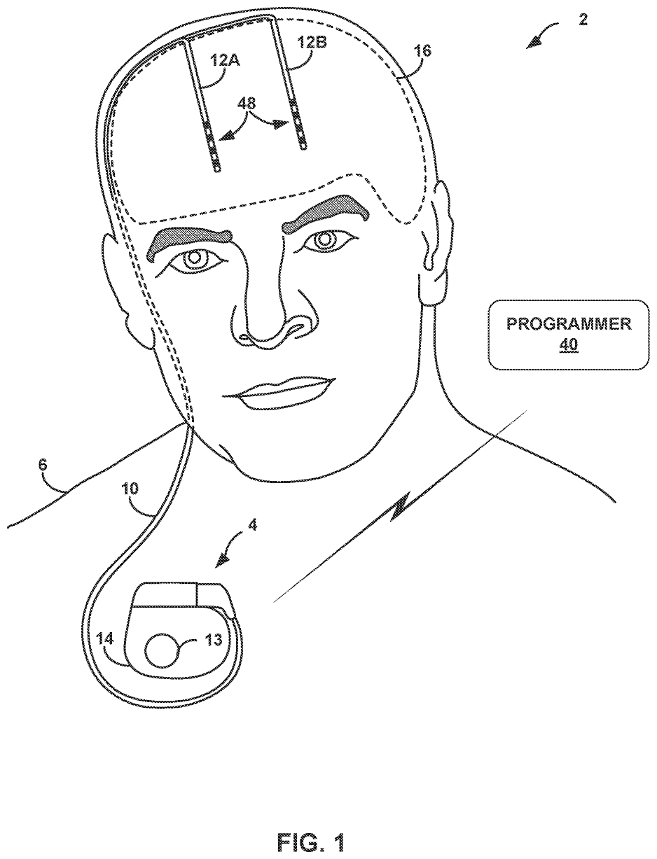

FIG. 1 is a conceptual diagram illustrating example therapy system 2 that includes electrical stimulator 4 coupled to stimulation lead 10, in accordance with various techniques of this disclosure. Therapy system 2 may be configured to deliver stimulation therapy to patient 6. Patient 6 ordinarily, but not necessarily, will be a human. Generally, therapy system 2 includes electrical stimulator 4 (e.g., an implantable medical device (IMD)) that delivers electrical stimulation to patient 6 via one or more electrodes disposed on stimulation lead extension 10. Electrical stimulator 4 delivers stimulation therapy, e.g., in the form of electrical stimulation, via one or more electrodes 48 disposed along one or more medical leads 12A and 12B which connect to lead extension 10. For purposes of description electrodes 48 are described as being implantable electrodes. However, the example techniques are not limited to implantable electrodes.

Electrodes 48 may be deployed on one or more medical leads, such as medical leads 12A and 12B, and in some cases on a housing electrode. The electrical stimulation may be in the form of controlled current pulses or voltage pulses, or substantially continuous current or voltage waveforms. A stimulation program may define various parameters of the pulses or waveforms. The pulses or waveforms may be delivered substantially continuously or in bursts, segments, or patterns, and may be delivered alone or in combination with pulses or waveforms defined by one or more other stimulation programs. In some examples, one or more of the electrodes may be located on a housing 14 of the electrical stimulator 4. In addition, implantable electrodes may be deployed on a leadless stimulator.

In some examples, electrical stimulator 4 may deliver, for example, deep brain stimulation (DBS) or cortical stimulation (CS) therapy to patient 6 via the electrodes carried by lead 12. Although FIG. 1 shows a particular stimulation environment (e.g., DBS), the techniques of this disclosure are not so limited, and electrical stimulator 4 may deliver stimulation therapy to other parts of patient 6, such as the spinal cord of patient 6. For example, other electrical stimulation systems may be configured to deliver electrical stimulation to gastrointestinal organs, the spinal cord, pelvic nerves or muscle, peripheral nerves, or other stimulation sites. In addition, although FIG. 1 shows a fully implantable electrical stimulator 4, techniques described in this disclosure may be applied to external stimulators having electrodes deployed via percutaneous leads.

In the example illustrated in FIG. 1, electrical stimulator 4 is implanted in a clavicle region of patient 6. Electrical stimulator 4 generates programmable electrical stimulation (e.g., a current or voltage waveform or current or voltage pulses) and delivers the stimulation via a medical lead 10 carrying an array of stimulation electrodes 48. In general, delivery of electrical stimulation using controlled current pulses will be described in this disclosure for purposes of illustration. In some cases, electrical stimulator may include multiple leads. In the example of FIG. 1, a distal end of lead 10 is bifurcated and includes two leads 12A and 12B (collectively “leads 12”). Leads 12A and 12B each include a set of electrodes forming part of the array of electrodes 48. In various examples, leads 12A and 12B may each carry four, eight, or sixteen electrodes. In FIG. 1, each lead 12A, 12B carries four electrodes, configured as ring electrodes at different axial positions near the distal ends of the leads 12.

In other examples, one or more of leads 12A or 12B may include a different array of electrodes, such as a complex electrode array geometry. For example, lead 12A may include electrodes at different positions around the perimeter of the lead. In one example, three, four, or more electrodes may be at the same axial position but different circumferential positions around the lead. These electrodes at different circumferential positions may be referred to as “segmented electrodes” because they represent “segments” of a ring around the lead. These electrodes at the same axial position may be referred to as being disposed at the same “level” of the lead. In some examples, a lead may include one or more levels of multiple electrodes and may include one or more complete ring (or cylindrical electrodes) in addition to the one or more levels of multiple electrodes. An example lead may include, from proximal to distal end of the lead, a proximal ring electrode, a first level of three electrodes, a second level of three electrodes, and a distal ring electrode. In other examples, a lead may include four levels of electrodes, where each level has two, three, four, or more electrodes. The electrodes may be circumferentially aligned or offset between levels.

FIG. 1 further depicts a housing electrode 13. Housing electrode 13 may be formed integrally with an outer surface of hermetically-sealed housing 14 of electrical stimulator 4, or otherwise coupled to housing 14. In one example, housing electrode 13 may be described as an active, non-detachable electrode on electrical stimulator 4. In some examples, housing electrode 13 is defined by an uninsulated portion of an outward facing portion of housing 14 of electrical stimulator 4. Other divisions between insulated and uninsulated portions of housing 14 may be employed to define two or more housing electrodes. In some examples, housing electrode 13 comprises substantially all of housing 14, one side of housing 14, a portion of housing 14, or multiple portions of housing 14.

In some examples, electrical stimulator 4 may be coupled to one or more leads which may or may not be bifurcated. In such examples, the leads may be coupled to electrical stimulator 4 directly or via a common lead extension (such as lead extension 10) or separate lead extensions. A proximal end of lead extension 10 may be coupled to a header on electrical stimulator 4. Conductors in the lead body may electrically connect stimulation electrodes located on leads 12 to electrical stimulator 4. Lead extension 10 traverses from the implant site of electrical stimulator 4 along the neck of patient 6 before coupling to leads 12A and 12B. Leads 12A and 12B continue to traverse to the brain 16 of patient 6. In some examples, leads 12A and 12B may be implanted within the right and left hemispheres, respectively, in order to deliver electrical stimulation to one more regions of brain 16.

Leads 12A, 12B may be implanted within a desired location of brain 16 through respective holes in the cranium of patient 6. Leads 12A, 12B may be placed at any location within brain 16 such that the electrodes located on leads 12A, 12B are capable of providing electrical stimulation to targeted tissue. The electrodes of leads 12A, 12B are shown as ring electrodes. In some examples, the electrodes of leads 12A, 12B may have different configurations. For example, the electrodes of leads 12A, 12B may have a complex electrode array geometry that is capable of producing shaped electrical fields. The complex electrode array geometry may include multiple electrodes (e.g., partial ring or electrode “segments”) around the perimeter of each leads 12A, 12B. In some examples, leads 12 may have shapes other than elongated cylinders as shown in FIG. 1. For example, leads 12 may be paddle leads, spherical leads, bendable leads, or any other type of shape effective in treating patient 6. In addition, the electrodes may be electrode pads on a paddle lead, circular electrodes surrounding the body of a lead, conformable electrodes, cuff electrodes, segmented electrodes, or any other type of electrodes capable of forming unipolar, bipolar, multi-polar, etc. electrode configurations.

In some examples, electrical stimulator 4 delivers stimulation according to a group of programs at a given time. Each program of such a program group may include respective values for each of a plurality of therapy parameters. The therapy parameters may include, e.g., one of a current or a voltage amplitude, a pulse width, a pulse shape, a pulse rate or pulse frequency, a number of pulses, or an electrode configuration (e.g., electrode combination and polarity). Electrical stimulator 4 may interleave pulses or other signals according to the different programs of a program group. In such examples, programmer 40 may be used to create programs, and assemble the programs into program groups. In some examples, programmer 40 may be used to adjust stimulation parameters of one or more programs of a program group, and select a program group as the current program group to control delivery of stimulation by electrical stimulator 4.

Generally, system 2 delivers stimulation therapy to patient 6 in the form of constant current or voltage waveforms or constant current or voltage pulses. The shapes of the pulses may vary according to different design objectives, and may include ramped or trapezoidal pulses, sinusoidal or otherwise curved pulses, stepped pulses having two or more discrete amplitudes, closely spaced pairs of pulses, and biphasic (positive and negative aspects within a single pulse) or monophasic (only positive or only negative aspects within a single pulse) variations of any of the above. In the case of current-based stimulation, electrical stimulator 4 regulates current that is sourced or sunk by one or more electrodes, referred to as regulated electrodes. In some examples, one or more of the electrodes may be unregulated. In such configurations, the housing electrode and/or a lead electrode may be the unregulated electrode. For example, in unipolar electrode combinations, the one or more anodes may be unregulated in the sense that they sink current delivered from the regulated cathodes. For bipolar electrode combinations, both anodes and cathodes may be regulated.

A source current may refer to a positive current that flows out of an electrode (anode), whereas a sink current may refer to a negative current that flows into an electrode (cathode). Regulated source currents may sum to produce a greater overall source current (e.g., currents from a plurality of source currents sum together to generate the overall source current). Likewise, regulated sink currents may sum to produce a greater overall sink current (e.g., currents from a plurality of sink currents sum together to generate the overall sink current). Regulated source and regulated sink currents may partially or entirely cancel one another, producing a net difference in the form of a net source current or sink current in the case of partial cancellation. In some examples, an unregulated current path can source or sink current approximately equal to this net difference. In some examples, regulated source and sink currents may be substantially balanced.

In some example implementations (e.g., bipolar (or multipolar) arrangements), one or more electrodes 48 may be configured to act as anodes and source current while one or more different electrodes 48 may be configured to act as cathodes and sink current. In another example implementation (e.g., unipolar arrangements), housing electrode 13 (e.g., a remote electrode from the target tissue) may be configured to act as an anode and source current while one or more electrodes 48 on one or more leads generally located near the target tissue are configured to act as cathodes and sink current. The techniques of this disclosure may be implemented using unipolar arrangements or bipolar/multipolar arrangements as described.

Therapy system 2 may include a programmer 40, such as an external programmer operated by a clinician or patient. In some examples, a programmer 40 may be a handheld computing device that permits a clinician to program stimulation therapy for patient 6 via a user interface. For example, using programmer 40, the clinician may specify stimulation parameters for use in delivery of stimulation therapy, and receive user request to convert bipolar electrode combinations to unipolar electrode combinations, and vice versa. Programmer 40 may support telemetry with electrical stimulator 4 to download programs and, optionally, upload operational or physiological data stored by electrical stimulator 4. Programmer 40 may also include a display and input keys to allow patient 6 or a clinician to interact with programmer 40 and electrical stimulator 4. In this manner, programmer 40 provides patient 6 with a user interface for control of the stimulation therapy delivered by electrical stimulator 4. For example, patient 6 may use programmer 40 to start, stop or adjust electrical stimulation. In particular, programmer 40 may permit patient 6 to adjust stimulation parameters of a program, such as duration, current or voltage amplitude, pulse width, pulse shape, and pulse rate. Patient 6 may also select a program (e.g., from among a plurality of stored programs) as the present program to control delivery of stimulation by electrical stimulator 4.

In some cases, programmer 40 may be characterized as a physician or clinician programmer 40. For example, programmer 40 may include a clinician programmer if programmer 40 is primarily intended for use by a physician or clinician. In other cases, programmer 40 may be characterized as a patient programmer if programmer 40 is primarily intended for use by a patient. In general, a physician or clinician programmer may support selection and generation of programs by a clinician for use by stimulator 4, whereas a patient programmer may support, during ordinary use, adjustment and selection by a patient of such programs as allowed by the clinician and/or clinician programmer.

Whether programmer 40 is configured for clinician or patient use, programmer 40 may communicate with electrical stimulator 4 or any other computing device via wireless communication. Programmer 40, for example, may communicate via wireless communication with electrical stimulator 4 using RF telemetry techniques known in the art. Programmer 40 may also communicate with another programmer or computing device via a wired or wireless connection using any of a variety of local wireless communication techniques, such as radio frequency (RF) communication according to the 802.11 or Bluetooth specification sets, infrared communication according to the Infrared Data Association (IrDA) specification set, or other standard or proprietary telemetry protocols. Programmer 40 may also communicate with another programming or computing device via exchange of removable media, such as magnetic or optical disks, or memory cards or sticks. Further, programmer 40 may communicate with electrical stimulator 4 and other programming devices via remote telemetry techniques known in the art, communicating via a local area network (LAN), wide area network (WAN), public switched telephone network (PSTN), or cellular telephone network, for example.

A user, such as a clinician or patient 6, may interact with a user interface of programmer 40 to program electrical stimulator 4. In accordance with various techniques described in this disclosure, programmer 40 may be used to receive user input, via the user interface specifying one or more therapy parameters for defining electrical stimulation therapy delivered by electrical stimulator 4. Programmer 40 may control electrical stimulator 4 to cause electrical stimulator 4 to deliver electrical stimulation therapy in accordance with the specified therapy parameters, as described in more detail below, or otherwise program stimulator 4. Programming of electrical stimulator 4 may refer generally to the generation and transfer of commands, programs, or other information to control the operation of electrical stimulator 4. For example, programmer 40 may transmit programs, parameter adjustments (such as electrode combinations), program selections, group selections, or other information to control the operation of electrical stimulator 4. In addition, programming of stimulator 4 may include receiving, via programmer 40, user input indicating a target stimulation zone or bipolar electrode combination, or request to convert a bipolar electrode combination to unipolar electrode combination.

Electrical stimulator 4 and programmer 40 may communicate via cables or a wireless communication, as shown in FIG. 1. Programmer 40 may, for example, communicate via wireless communication with electrical stimulator 4 using RF telemetry techniques. Programmer 40 may also communicate with other programmers using any of a variety of local wireless communication techniques, such as RF communication according to the 802. 11 or Bluetooth™ specification sets, infrared communication (e.g., according to the IrDA standard), or other standard or proprietary telemetry protocols. Programmer 40 may include a transceiver to permit bi-directional communication with electrical stimulator 4.

In accordance with the techniques of the disclosure, system 2 can include processing circuitry configured to convert bipolar electrode combinations to unipolar electrode combinations, and vice versa. A bipolar electrode combination may be an electrode combination with one or more anodes and one or more cathodes on the same lead or one or more leads disposed near the target tissue. A bipolar electrode combination may also be referred to as a multipolar electrode combination. In a bipolar electrode combination, electrical current flows a relatively short distance between the anodes and cathodes of the bipolar electrode combination. In some examples, a bipolar electrode combination includes regulated anodes and regulated cathodes to control the current that is sourced and sunk to each electrode. A unipolar electrode combination includes one or more electrodes near target tissue of one polarity, such as cathodes, and one or more electrodes remote from the target tissue such that the target tissue is generally affected by the single polarity electrodes close by. In some examples, the close electrodes may be regulated, such as one or more cathodes, and are sunk to one or more unregulated anodes remote from the target tissue.

System 2 may be configured to receive a first stimulation parameter set comprising a bipolar electrode combination that defines a first electrical stimulation. This bipolar electrode combination may be selected by a user or automatically selected based on instructions stored in memory or otherwise programmed. System 2 may estimate a volume of neural activation (VNA) corresponding to the first stimulation parameter set. The VNA may be modeled based on the bipolar electrode combination, other stimulation parameter values, and tissue characteristics that may be generic or patient-specific. System 2 may then determine, based on the first VNA, a second stimulation parameter set that includes a unipolar electrode combination that defines a second electrical stimulation. The second stimulation parameter set that includes the unipolar electrode combination may be associated with a second VNA that may be similar to or approximate to the first VNA. In other words, system 2 may be configured to identify a unipolar electrode combination and other parameter values that may generate a VNA similar to the VNA of the bipolar electrode combination. Then, system 2 can control IMD 14 to deliver the second electrical stimulation from the unipolar electrode combination instead of the first electrical stimulation from the bipolar electrode combination. Both of the first stimulation parameter set and the second stimulation parameter set includes respective at least one of current or voltage amplitude, a pulse width, and a pulse frequency. In some examples, the first and second parameter sets may be identical except for the respective unipolar and bipolar electrode combinations. In other examples, the first and second parameter sets may have one or more different parameter values, such as a different amplitude or pulse width, for example.

In some examples, system 2 may be configured to convert the bipolar electrode combination to a unipolar electrode combination based on an estimated VNA for the bipolar electrode combination and VNA for possible corresponding unipolar electrode combinations. System 2 may attempt to identify VNAs that will be similar for unipolar electrode combinations than for bipolar electrode combinations. In one example, system 2 can selecting one or more cathodes on a lead that carried the bipolar electrode combination. As discussed herein, the bipolar electrode combination may include electrodes on the same lead or on one or more leads that are adjacent to the target tissue. In contrast, the unipolar electrode combination may include one or more cathodes on the one or more leads (such as the leads that carried the bipolar electrode combination) and at least one anode remote from the one or more lead and the target tissue, such as an electrode on the housing of IMD 14.

System 2 may be configured to estimate the VNA corresponding to stimulation generated by one or more unipolar electrode combinations. For example, system 2 may be configured to analyze different unipolar electrode combinations that are associated with VNAs having a similar shape and/or volume to that of the VNA of the bipolar electrode combination. System 2 may also be configured to identify an amplitude value for the one or more cathodes at which one or more characteristics of the second VNA corresponds to one or more characteristics of the first VNA. The amplitude value at each cathode or anode may correspond to the distance that the VNA reaches out away from the respective electrodes. In other words, greater amplitudes may generally result in a larger VNA that extends a greater radius away from the electrode, The characteristics of the VNA that may be compared may include volume, shape, radius of the VNA from one or more of the electrodes, etc. Once system 2 determines the appropriate unipolar electrode combination that has a VNA similar to the VNA of the bipolar electrode combination, system 2 may generate the second stimulation parameter set to include the one or more cathodes and the amplitude value. In some examples, the amplitude value of at least one, or up to all, of the electrodes of the unipolar electrode combination may be less than the amplitude value of the first stimulation parameter set and bipolar electrode combination.

In some examples, system 2 may be configured to select one or more cathodes on the lead for the unipolar electrode combination by keeping any cathodes of the bipolar electrode combination and switching any anodes of the bipolar electrode combination to cathodes. In this manner, all of the cathodes would be using the same electrodes on the leads as the bipolar electrode combination. In addition, system 2 may add one or more anodes remote from the leads, such as an electrode on the housing of IMD 14.

As described above, each VNA may include one or more characteristics that system 2 can compare to other VNAs in order to identify unipolar electrode combinations that may be appropriate alternatives to a bipolar electrode combination. In some examples the VNA characteristics may include one or more of a volume, a distance from a respective electrode, or a distance from the lead at a respective axial position. System 2 may be configured to identify the amplitude value for the one or more cathodes, or one or more anodes, at which one or more characteristics of the second VNA corresponds to one or more characteristics of the first VNA. In some examples, some characteristics of the VNA may be weighted differently for the purposes of comparison and identifying a suitable unipolar electrode combination for conversion from the bipolar electrode combination. In other words, characteristics that are more important to identifying a similar VNA may be weighted higher than other characteristics that may be less of a factor to the patient. For example, the distance the VNA extends radially from the electrodes may be of more importance, and weighted higher, than the overall volume of the VNA. Therefore, system 2 may determine the best VNA, and corresponding best unipolar electrode combination, and other parameter values, based on these weighted characteristics of the VNAs. In one example, the system may identify the amplitude value for the one or more cathodes of the unipolar electrode combination based on differently weighted characteristics of the one or more characteristics of the first VNA of the bipolar electrode combination.

In some examples, system 2 may only identify a single VNA and corresponding unipolar electrode combination. In other examples, system 2 may generate multiple different VNAs for one or more unipolar electrode combinations as candidate VNAs to compare the VNA of the bipolar electrode combination. Then, system 2 may compare the VNAs to identify the closest, or best fit, VNA of the unipolar electrode combinations to the bipolar VNA. In some examples, system 2 may present different options of VNAs, which are generated for different unipolar electrode combinations and/or other parameter values to the user for selection. System 2 may then receive user input selecting the desired VNA or other representations of the parameter values associated with the desired VNA. In some examples, system 2 may rank the candidate VNAs or candidate parameter values based on the fit to the VNA of the bipolar electrode combination.

In some examples, the bipolar electrode combination includes at least one anode disposed on an implantable medical lead, such as leads 12A and 12B, and at least one cathode disposed on the implantable medical leads, such as leads 12A or 12B. The unipolar electrode combination may include at least one cathode disposed on an implantable medical lead, such as leads 12A and 12B, and at least one anode disposed on IMD 14, for example. In some examples, the bipolar electrode combination includes at least one electrode of a plurality of electrodes disposed at different locations around a perimeter of a lead.

System 2 may be configured to receive user input requesting to convert a bipolar electrode combination to a unipolar electrode combination. For example, programmer 40 may include a display, where processing circuitry is configured to control the display to present a selectable icon that, when selected, causes the processing circuitry to determine, based on the first VNA of the bipolar electrode combination, the second stimulation parameter set comprising the unipolar electrode combination that defines the second electrical stimulation.

In some examples, system 2 may be configured to control a user interface, such as a user interface presented by programmer 40, to present a representation of first electrical stimulation defined by a first stimulation parameter set comprising a bipolar electrode combination. This representation may be in the form of a graphical VNA, numerical characterization of one or more characteristics of the VNA (e.g., radial distance from electrodes or volume), or the like. System 2 may control the user interface to present a selectable icon associated with converting the first electrical stimulation defined by the first stimulation parameter set comprising the bipolar electrode combination to a second electrical stimulation defined by a second stimulation parameter set comprising a unipolar electrode combination. In other words, selection of the selectable icon may trigger system 2 to convert the bipolar electrode combination to a unipolar electrode combination, or vice versa. System 2 may then receive user selection of the selectable icon and, responsive to receiving the user selection of the selectable icon, converting the first electrical stimulation to the second electrical stimulation defined by the second stimulation parameter set comprising the unipolar electrode combination. System 2 may control the user interface of programmer 2 to present a visual representation of the VNA corresponding to the electrical stimulation of the bipolar electrode combination and the unipolar electrode combination separately, next to each other, or overlapping to illustrate differences between the VNAs. In some examples, system 2 can control the user interface to present one or more controls configured to receive user input adjusting a value of respective stimulation parameters of the second stimulation parameter set. Adjustable parameters may include an amplitude, pulse width, or frequency. In some examples, user input may also be received to add or remove electrodes to the electrode combination and/or change electrodes from anode to cathode or cathode to anode.

In some examples, selection of the selectable icon (e.g., a convert button) may cause system 2 to switch from a bipolar electrode combination, and back again, in response to each selection. In this manner, system 2 may be configured to toggle between the first electrical stimulation comprising the bipolar electrode combination and the second electrical stimulation comprising the unipolar electrode combination in response to user selection of the selectable icon.

In some examples, programmer 40 (e.g., a clinician or patient programmer) may include the processing circuitry and the memory for performing the functions described herein, such as converting a bipolar electrode combination to a unipolar electrode combination. In other examples, another device, such as IMD 14 or an external server, may include the processing circuitry and memory for performing these functions, or a combination of two or more devices may perform aspects of the functions in a distributed manner.

The techniques of the disclosure may provide specific improvements to the computer-related field of neurostimulation therapy that have practical applications. For example, the techniques described herein enable a user, or a system, to determine unipolar electrode combination alternatives from a bipolar electrode combination identified to provide therapy to a patient. In this manner, the unipolar electrode combination may be used to deliver electrical stimulation that consumes less power, and increases battery longevity, when compared to stimulation delivered from the original bipolar electrode combination. In addition, the techniques described herein enable a user interface to receive user requests to change, or convert, from a bipolar electrode combination to a unipolar electrode combination that can produce a similar VNA for treating the patient. In this manner, the user interface may reduce programming time while identifying alternative parameter sets that may reduce power consumption.

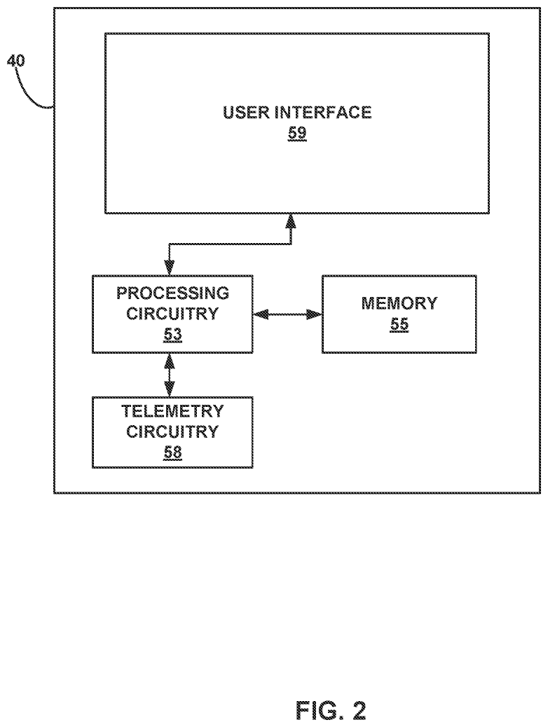

FIG. 2 is a block diagram illustrating example programmer 40 of FIG. 1 in further detail. As shown in FIG. 2, programmer 40 includes processing circuitry 53, memory 55, telemetry circuitry 58, and user interface 59. In general, processing circuitry 53 controls user interface 59, stores and retrieves data to and from memory 55, and controls transmission of data with electrical stimulator 4 through telemetry circuitry 58. Processing circuitry 53 may take the form of one or more microprocessors, controllers, digital signal processors (DSPs), application-specific integrated circuits (ASICs), field-programmable gate arrays (FPGAs), or equivalent discrete or integrated logic circuitry. The functions attributed to processing circuitry 53 herein may be embodied as software, firmware, hardware or any combination thereof.

Memory 55 may store instructions that cause processing circuitry 53 to provide various aspects of the functionality ascribed to programmer 40 herein. Memory 55 may include any fixed or removable magnetic, optical, or electrical media, such as random access memory (RAM), read-only memory (ROM), compact disc ROM (CD-ROM), magnetic memory, electronically-erasable programmable ROM (EEPROM), non-volatile random access memory (NVRAM), flash memory, etc. Memory 55 may also include a removable memory portion that may be used to provide memory updates or increases in memory capacities. A removable memory may also allow patient data to be easily transferred from programmer 40 to another computing device. Memory 55 may also store information that controls operation of electrical stimulator 4.

Telemetry circuitry 58 is configured to transfer data to and from electrical stimulator 4. Telemetry circuitry 58 may communicate automatically with electrical stimulator 4 at a scheduled time or when telemetry circuitry 58 detects the proximity of electrical stimulator 4. Alternatively, telemetry circuitry 58 may communicate with electrical stimulator 4 when signaled by a user through user interface 59. To support RF communication, telemetry circuitry 58 may include appropriate electronic components, such as amplifiers, filters, mixers, encoders, decoders, etc. In some examples, programmer 40 may communicate wirelessly with electrical stimulator 4 using, for example, RF communication or proximal inductive interaction. This wireless communication is possible through the use of telemetry circuitry 58 which may be coupled to an antenna. Programmer 40 may also be configured to communicate with another computing device via wireless communication techniques, or direct communication through a wired, e.g., network, connection. Examples of local wireless communication techniques that may be employed to facilitate communication between programmer 40 and another computing device include RF communication based on the 802.11 or Bluetooth specification sets, infrared communication.

Programmer 40 includes user interface 59. A user (e.g., a clinician or patient 6) may interact with programmer 40 via user interface 59 to, for example, manually select, change or modify programs, adjust one or more therapy parameters of specific electrodes 48 or a plurality of electrodes 48, or view stimulation data. As another example, user interface 59 may display VNAs, electrodes that are selected, a convert button selectable by the user to switch to unipolar electrode combinations from a bipolar electrode combination, etc. User interface 59 may comprise one or more input devices and one or more output devices. The input devices of user interface 59 may include a communication device such as a keyboard, pointing device, voice responsive system, video camera, biometric detection/response system, button, sensor, control pad, microphone, presence-sensitive screen, or any other type of device for detecting input from the user.

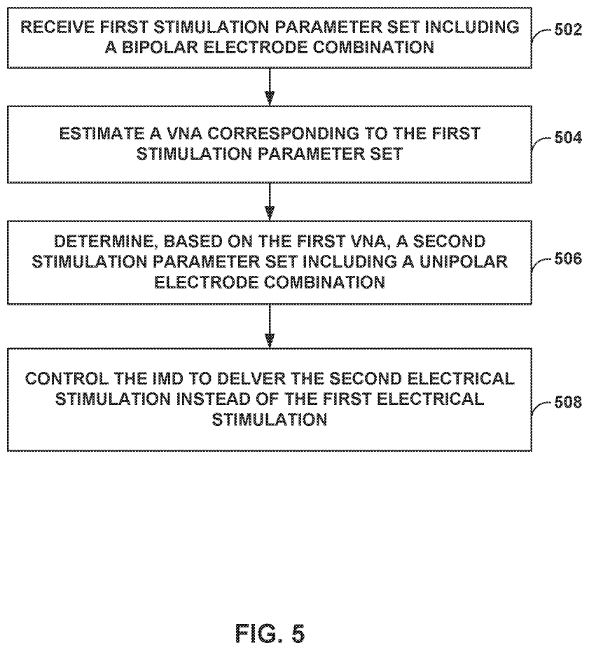

The output devices of user interface 59 may include a communication unit such as a display, sound card, video graphics adapter card, speaker, presence-sensitive screen, one or more USB interfaces, video and/or audio output interfaces, or any other type of device capable of generating tactile, audio, video, or other output. The output devices of user interface 59 may include a display device, which may function as an output device using technologies including liquid crystal displays (LCD), quantum dot display, dot matrix displays, light emitting diode (LED) displays, organic light-emitting diode (OLED) displays, cathode ray tube (CRT) displays, e-ink, or monochrome, color, or any other type of display capable of generating tactile, audio, and/or visual output. In other examples, the output devices of user interface 59 may produce an output to a user in another fashion, such as via a sound card, video graphics adapter card, speaker, presence-sensitive screen, one or more USB interfaces, video and/or audio output interfaces, or any other type of device capable of generating tactile, audio, video, or other output. In some examples, the output devices of user interface 59 may include a presence-sensitive display that may serve as a user interface device that operates both as one or more input devices and one or more output devices. Additional detail regarding an example of user interface 59 is described with respect to FIGS. 5, 6, 7A, and 7B below.

In accordance with the example techniques of the disclosure, user interface 59 presents a representation of the plurality of electrodes 48 of electrical stimulator 4. User interface 59 presents, e.g., values of the therapy parameters specific to each of the plurality of electrodes 48, a selection of the multiple electrodes 48 for which the relationship is defined (e.g., anodes and cathodes of bipolar or unipolar electrode combinations), a convert button selectable by the user, etc. User interface 59 may provide fillable fields, or other adjustment input devices, such as increase or decrease input keys, that allow a user to input a desired value for a therapy parameter of a given electrode 48 targeted for adjustment, for multiple electrodes 48 targeted for adjustment, or a master adjustment of electrical stimulator 4. Processing circuitry 53 may receive, from the user via user interface 59, an input specifying adjustments to the values of the individual therapy parameters by receiving an input specifying an increase or decrease in the corresponding value of the parameter displayed on user interface 59. Further, processing circuitry 53 may receive, from the user, an adjustment to one or more of the therapy parameters corresponding to each particular electrode 48 by receiving an input specifying an increase or decrease in a value of the one or more therapy parameters displayed on the user interface of programmer 40.

In operation, processing circuitry 53 receives, from the user via user interface 59, a request to adjust one or more of therapy parameters corresponding to each particular electrode 48. For example, processing circuitry 53 may receive an input specifying an increase or decrease in a value of the one or more therapy parameters displayed on the user interface of programmer 40, such as amplitude, pulse width, frequency, or polarity (cathode or anode). Processing circuitry 53 transmits, via telemetry circuitry 58, the specified individual therapy parameters to electrical stimulation 4 to control electrical stimulator 4 to deliver, via multiple electrodes 48, electrical stimulation therapy according to respective therapy parameters of each of the multiple electrodes 48.

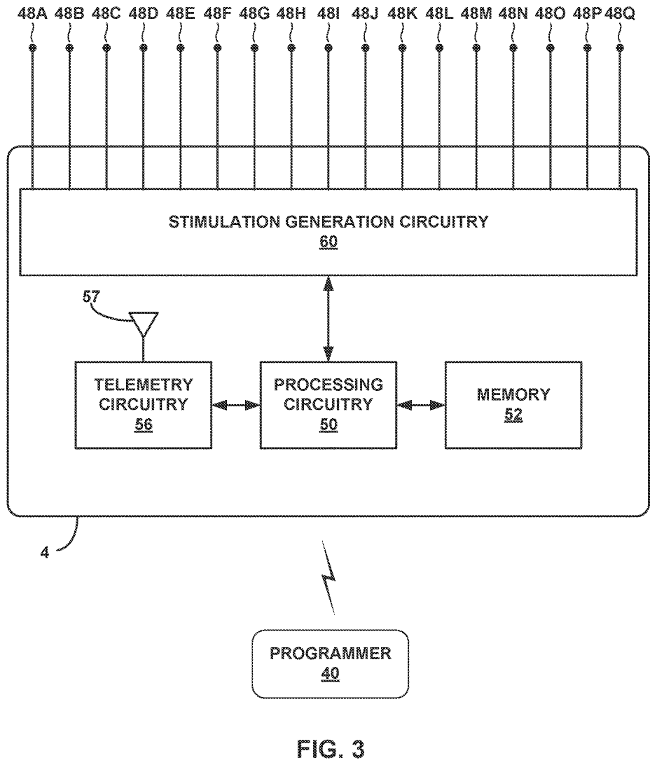

FIG. 3 is a block diagram illustrating example electrical stimulator 4 of FIG. 1 in further detail. In some examples, electrical stimulator 4 includes processing circuitry 50, memory 52, telemetry circuitry 56, antenna 57, and stimulation generation circuitry 60. Stimulation generation circuitry 60 is also shown in FIG. 3 coupled to electrodes 48A-Q (collectively “electrodes 48”). In some examples, electrodes 48A-48P may be implantable and may be deployed on one or more leads 12. With respect to FIG. 1, leads 12A and 12B may carry electrodes 48A-H and electrodes 48I-P, respectively. In some cases, one or more additional electrodes may be located on or within the housing of electrical stimulator 4, e.g., to provide a common or ground electrode or a housing anode. In some examples, a lead or lead carries eight electrodes to provide a 2×8 electrode configuration (two leads with 8 electrodes each), providing a total of sixteen different electrodes.

In some examples, different electrode configurations comprising a single lead, two leads, three leads, or more may be provided. In addition, electrode counts on leads may vary and may be the same or different from a lead to lead. Examples of other configurations include one lead with eight electrodes (1×8), one lead with 12 electrodes (1×12), one lead with 16 electrodes (1×16), two leads with four electrodes each (2×4), three leads with four electrodes each (3×4), three leads with eight electrodes each (3×8), three leads with four, eight, and four electrodes, respectively (4-8-4), two leads with 12 or 16 electrodes (2×12, 2×16), two or more leads with 11 or 13 electrodes, or other configurations. Processing circuitry 50 may select different electrodes to form various electrode combinations. In addition, processing circuitry 50 may assign various polarities to the selected electrodes to designate the electrodes as anodes or cathodes and form additional electrode configurations therefrom. Fewer or greater electrodes may be controlled by electrical stimulator 4 in other examples. For example, stimulation generation circuitry 60 may be coupled to 16 electrodes, 8 electrodes on each of two leads. For example each lead may include two ring electrodes and two levels of three electrodes at different circumferential positions around the lead perimeter.

Electrode 48Q represents one or more electrodes that may be carried on a housing of electrical stimulator 4. Electrode 48Q may also be a dedicated short lead extending from the housing, or a proximal portion of one of the leads carrying electrodes 48A-48P. The proximal portion may be closely adjacent to the housing, e.g., at or near a point at which a lead is coupled to the housing. Electrode 48Q may be configured as a regulated or unregulated electrode for use in an electrode configuration with selected regulated and/or unregulated electrodes among electrodes 48A-48P, which may be located on a lead body of one or more leads, as described above. Electrode 48Q may be formed together on a housing that carries the electrode and houses the components of electrical stimulator 4, such as stimulation generation circuitry 60, processing circuitry 50, memory 52, and telemetry circuitry 56.

Housing electrode 48Q may be configured for use as an anode to source current substantially simultaneously with one or more electrodes 48A-48P configured for use as cathodes sinking current in a unipolar arrangement. By way of specific example, electrodes 48A, 48B, and housing electrode 48Q each could be configured for use as anodes. Electrodes 48A, 48B could deliver electrical stimulation current substantially simultaneously with the electrical stimulation current delivered via housing electrode 48Q. In this illustration, one or more cathodes could be formed with other electrodes (e.g., any of electrodes 48C-48P) on the leads to sink current sourced by anodes 48A, 48B and 48Q.

Memory 52 may store instructions for execution by processing circuitry 50, stimulation therapy data, sensor data, instructions for converting bipolar electrode combinations to unipolar electrode combinations, and/or other information regarding therapy for patient 6. Processing circuitry 50 may control stimulation generation circuitry 60 to deliver stimulation according to a selected one or more of a plurality of programs or program groups stored in memory 52. Memory 52 may include any electronic data storage media, such as RAM, ROM, EEPROM, NVRAM, flash memory, magnetic memory, or the like. Memory 52 may store program instructions that, when executed by processing circuitry 50, cause the processing circuitry to perform various functions ascribed to processing circuitry 50 and electrical stimulator 4 in this disclosure.

Processing circuitry 50 may include one or more microprocessors, DSPs, ASICs, FPGAs, or other digital logic circuitry. Processing circuitry 50 controls operation of electrical stimulator 4. For example, processing circuitry 50 may control stimulation generation circuitry 60 to deliver stimulation therapy according to a selected program or group of programs retrieved from memory 52. In some examples, processing circuitry 50 may control stimulation generation circuitry 60 to deliver electrical signals, e.g., as stimulation pulses or continuous waveforms, with current amplitudes, pulse widths (if applicable), and rates specified by one or more stimulation programs. Processing circuitry 50 may also control stimulation generation circuitry 60 to selectively deliver stimulation via subsets of electrodes 48, also referred to as electrode combinations, and with polarities specified by one or more programs. The functions attributed to processing circuitry 50 herein may be embodied as software, firmware, hardware or any combination thereof.

Upon selection of a particular program group, processing circuitry 50 may control stimulation generation circuitry 60 to deliver stimulation according to programs in the groups. Each program may specify a set of stimulation parameters, such as amplitude, pulse width, pulse rate, and electrode combination, if applicable. For a continuous waveform, parameters may include amplitude and frequency. In addition, each program may specify a particular electrode combination for delivery of stimulation, and an electrode configuration in terms of the polarities and regulated/unregulated status of the electrodes. The electrode combination may specify particular electrodes in a single array or multiple arrays, and on a single lead or among multiple leads. The electrode combination may include at least one anode on the housing of the electrical stimulator 4 (e.g., electrode(s) 48Q), at least one anode on a lead, and at least one cathode on a lead. The lead-borne anode and cathode may be on the same lead or different leads, if more than one lead is provided. A program may be defined directly, by selecting parameters and electrodes, or by zone-based programming, in which parameters and electrodes are automatically determined by the programmer in response to manipulation or positioning of stimulation zones.

Stimulation generation circuitry 60 is electrically coupled to electrodes 48A-P via conductors of the respective lead, such as lead 12 in FIG. 1. Stimulation generation circuitry 60 may be electrically coupled to one or more housing electrodes 48Q via an electrical conductor disposed within the housing of electrical stimulator 4. Housing electrode 48Q may be configured as a regulated or unregulated electrode to form an electrode configuration in conjunction with one or more of electrodes 48A-48P. Housing electrode 48Q may be configured for use as an anode to source current substantially simultaneously with one or more electrodes, e.g., any of electrodes 48A-48P, on one or more leads configured for use as anodes. Stimulation generation circuitry 60 may include stimulation generation circuitry to generate stimulation pulses or waveforms and circuitry for switching stimulation across different electrode combinations, e.g., in response to control by processing circuitry 50. Stimulation generation circuitry 60 produces an electrical stimulation signal in accordance with a program based on control signals from processing circuitry 50.

In one example implementation, stimulation generation circuitry 60 may be configured to deliver stimulation using one or more of electrodes 48A-P and housing electrode 48Q as stimulation electrodes, e.g., anodes. The anodes on the lead(s) and the housing may be used to deliver stimulation in conjunction with one or more cathodes on the lead(s). As one illustration, an electrode combination selected for delivery of stimulation current may comprise a housing anode, and anode on a lead, and a cathode on the same lead or a different lead. In other examples, the electrode combination may include multiple anodes and/or multiple cathodes on one or more leads in conjunction with at least one anode on housing 14. In some examples, the electrode combination may include one or more anodes on one or more leads, and one or more cathodes on the same lead or a different lead, e.g., a bipolar/multipolar arrangement.

Telemetry circuitry 56 may include a RF transceiver to permit bi-directional communication between electrical stimulator 4 and programmer 40. Telemetry circuitry 56 may include an antenna 57 that may take on a variety of forms. For example, antenna 57 may be formed by a conductive coil or wire embedded in a housing associated with medical device 4. In some examples, antenna 57 may be mounted on a circuit board carrying other components of electrical stimulator 4 or take the form of a circuit trace on the circuit board. In this way, telemetry circuitry 56 may permit communication with programmer 40 in FIG. 1, to receive, for example, new programs or program groups, or adjustments to programs or program groups. Telemetry circuitry 56 may be similar to telemetry circuitry 58 of programmer 40.

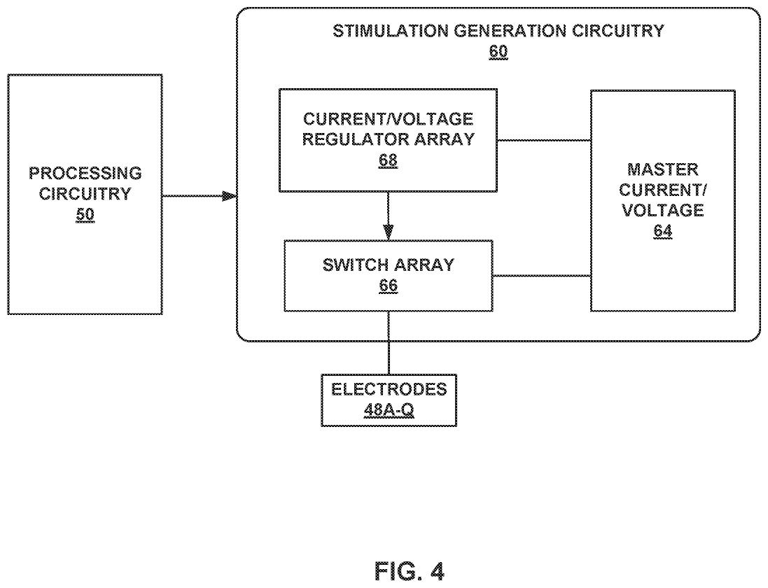

FIG. 4 is a block diagram illustrating an example of electrical stimulation generation circuitry 60 of electrical stimulator 4 of FIG. 3 in further detail. Stimulation generation circuitry 60 may be used with an electrical stimulator, e.g., to perform the functions of stimulation generation circuitry 60 as described with reference to FIG. 3. In the example of FIG. 4, stimulation generation circuitry 60 is selectively configured to deliver current stimulation pulses to patient 6 via electrodes 48. However, this disclosure is not limited to examples in which regulated current pulses are delivered. In other examples, stimulation generation circuitry 60 may provide continuous, regulated current waveforms, rather than regulated current pulses. In some examples, stimulation generation circuitry 60 may deliver combinations of continuous waveforms and pulses, or selectively deliver either continuous waveforms or pulses. Stimulation generation circuitry 60 may generate either constant current-based or constant voltage-based stimulation in the form of pulses or continuous waveforms. Stimulation generation circuitry 60 may also be controlled to provide constant power (current-voltage product) or controlled charge stimulation pulses.

In the example illustrated in FIG. 4, stimulation generation circuitry 60 includes master current/voltage 64, and current/voltage regulator array 68. In some examples, stimulation generation circuitry 60 may further include a switch array 66. Master current/voltage 64 may provide operating power to current/voltage regulator array 68, and may include a regulated current or regulated voltage that sets the level of the master current (e.g., master electrical current amplitude) or master voltage. As shown in FIG. 4, master current/voltage 64 may be coupled to provide operating power for the current/voltage regulator array 68 and provide a master current, or master voltage when appropriate, for connection to electrodes 48. The maximum operating current level and the master current level provided to regulate current regulator array 68 may be different at any given time For example, a master electrical current amplitude may be less than the maximum operating current level, such that the master electrical current amplitude may be increased or decreased according to minimum and maximum operating conditions. In some examples, user interface 59 of external programmer 40 may display such information for a user to reference while adjusting electrical current amplitudes for various electrodes.

Processing circuitry 50 may control (e.g., via a stimulation controller) switch array 66 and current/voltage regulator array 68 to deliver stimulation via electrodes 48. In operation, processing circuitry 50 may control delivery of electrical stimulation according to one or more programs that may specify stimulation parameters such as electrode combination, electrode polarity, stimulation current amplitude, pulse rate, and/or pulse width as well as the percentage of source current distributed among or contributed by a housing anode and one or more lead anodes on one or more leads, and the percentage of sink current sunk by one or more cathodes. Programs may be defined by a user via an external controller and downloaded to an electrical stimulator 4.

Current/voltage regulator array 68 includes a plurality of regulated current sources or sinks. A current regulator may function as either a current source or sink, or be selectively configured to operate as either a source or a sink. In some examples, current/voltage regulator array 68 may regulate voltage instead of, or in addition to, current. For convenience, the term “current regulator” may be used in some instances to refer to either a source or sink. Hence, each of the current regulators in current/voltage regulator array 68 may operate as a regulated current source that delivers stimulation via a corresponding one of electrodes 48 or a regulated current sink that receives current from a corresponding one of electrodes 48, where electrodes 48 may be provided on leads, on a stimulator housing, on a leadless stimulator, or in other arrangements. Although multiple current sources or sinks are described herein, electrical stimulator 4 may include a single current source or sink in other examples and still support locking multiple electrodes into a relationship having a ratio of values for one or more therapy parameters.

Each current regulator may correspond to a plurality of current regulator branches. In some examples, the current regulator branches may be implemented in a parallel, such as with parallel current regulator branches. The number of current regulator branches defines the resolution for each current regulator. For example, the number of current regulator branches may be 64 in some examples, such that the electrical current amplitude may be adjusted for a given electrode in 1/64 increments (i.e., a resolution of 1/64). While 64 current branches are used for example throughout this disclosure, the techniques of this disclosure are not so limited, and the number of current branches may be more or fewer than 64 branches. For example, in some implementations, 128 current branches may be used, such that the current regulator for a particular electrode may be adjusted in 1/128 increments (i.e., a resolution of 1/128). In an illustrative example implementation with a resolution of 1/64, a ring electrode at full output may implement 64branches (e.g., 64/64^{ths}). In addition, stimulation generation circuitry 60 may be set such that, for each of the highest contributing electrodes of the highest intensity active zone, all 64 parallel current regulator branches are used.

In examples involving leads with electrodes at different circumferential positions around the perimeter of the lead (e.g., segmented electrodes or a complex electrode array), electrodes at various axial positions of lead 12 may have a fraction maximum equal to approximately the number of branches available to the electrode divided by the number of electrodes segments in a ring of segmented electrodes. For example, ring electrodes may have a maximum of 64/64 fractions in an example involving 64 current regulator branches, whereas each of N segmented electrodes in a ring of segmented electrodes may have a maximum of approximately 64/N fractions. In an illustrative example, in the case of three segmented electrodes in a ring, each electrode may have a fraction maximum of 21/64 fractions. In some examples, the fraction maximum for any given electrode, including ring electrodes, may reach the full number of current regulator branches (e.g., 64 branches). That is, processing circuitry 53 or processing circuitry 50 may be configured to impose any fraction maximum based on the particular stimulation generation circuitry 60 in use (e.g., the number of current regulator branches). For example, in the case of three segmented electrodes in a ring as in the previous example, each electrode may have a fraction maximum of X/X fractions (e.g., 64/64 fractions) or a fraction less than X/X that has been predefined by processing circuitry 53 or processing circuitry 50.

In examples including switch array 66, each switch of switch array 66 may couple a corresponding one of electrodes 48 to either a corresponding bi-directional current regulator of current/voltage regulator array 68 or to master current/voltage 64. In some examples, processing circuitry 50 selectively opens and closes switches in switch array 66 to configure a housing electrode (e.g., electrode(s) 48Q), and one or more of electrodes 48A-48P on one or more leads as regulated electrodes by connection to regulated current sources or sinks in current/voltage regulator array 68. In some examples, processing circuitry 50 may selectively open and close switches in switch array 66 to configure either the housing electrode, e.g., electrode 48Q, or an electrode on the lead as an unregulated electrode by connection to master current/voltage 64. In addition, processing circuitry 50 may selectively control individual regulated current sources or sinks in current/voltage regulator array 68 to deliver stimulation current pulses to the selected electrodes. In examples where switch array 66 is not used, electrodes 48 may nevertheless be coupled to current/voltage regulator array 68 and/or to master current/voltage 64.

Master current/voltage 64 may be a high or low voltage supplied by a regulated power source, depending on whether an electrode is programmed to be an unregulated source (high voltage rail) or unregulated sink (low voltage rail). Hence, master current/voltage 64 may produce high and low master current, or master voltages when appropriate, for selective coupling to unregulated, reference electrodes as needed. A regulated power source may produce one or more regulated voltage levels for use as master current/voltage 64 and for use as a power rail for current/voltage regulator array 68. Although the same master current/voltage 64 is shown as being coupled to current/voltage regulator array 68 in FIG. 4, different current amplitude may be used for the master current coupled to switch array 66 and the maximum current amplitude provided to current regulator array 68. In any event, a regulated power source may generate the regulated current amplitudes from current provided by a power source or multiple power sources, such as one or more batteries (e.g., rechargeable batteries).

Processing circuitry 50 controls the operation of switch array 66 to produce electrode configurations defined by different stimulation programs. In some cases, the switches of switch array 66 may be metal-oxide-semiconductor field-effect-transistors (MOSFETs) or other circuit components used for switching electronic signals. The switches of switch array 66 may be designed to carry an amount of unregulated current that may be coupled to a corresponding electrode through an unregulated current path associated with master current/voltage 64. In some examples, two or more regulated electrodes 48 may be intentionally programmed to deliver different amounts of current, such that the regulated electrodes produce an unbalanced current distribution. In other examples, regulated source and sink current may be balanced such that substantially all current may be sourced and sunk via respective regulated current sources and sinks.

To provide individual control of electrodes 48 as either regulated electrodes or as unregulated, reference electrodes, processing circuitry 50 controls operation of switch array 66 and current/voltage regulator array 68. When stimulation is delivered to patient 6, for the example of current pulses, processing circuitry 50 controls switch array 66 to couple selected stimulation electrodes for a desired electrode combination to respective current regulators of current/voltage regulator array 68 or to master current/voltage 64, as needed. Processing circuitry 50 controls the regulated bi-directional current sources of current/voltage regulator array 68 coupled to regulated electrodes to source or sink specified amounts of current. For example, processing circuitry 50 may control selected current sources or sinks on a pulse-by-pulse basis to deliver current pulses to corresponding electrodes.

Processing circuitry 50 also deactivates the regulated bi-directional current regulators of current/voltage regulator array 68 tied to inactive electrodes, e.g., electrodes that are not active as regulated electrodes in a given electrode configuration. Each regulated bidirectional current regulator of current/voltage regulator array 68 may include an internal enable switch controlled by processing circuitry 50 that disconnects regulated power from the current regulator or otherwise disables the current source when the corresponding electrode is not used as a regulated electrode.

The use of stimulation generation circuitry 60 enables delivery of current in fractional amounts according to a fractional use of the current regulators of current/voltage regulator array 68 and switch array 66. In this fashion, electrical stimulator 4 may deliver electrical stimulation via each of electrodes 48 that has, e.g., a fractional current amplitude of a current amplitude of each other electrode 48. Thus, the use of stimulation generation circuitry 60 allows for the adjustment of therapy parameters defining electrical stimulation therapy delivered electrodes 48 while also allowing electrical stimulator 4 to maintain a ratio of values of each therapy parameter of each of the electrodes 48 to one another.

For example, external programmer 40 of FIG. 1 defines a relationship for multiple electrodes of a plurality of electrodes 48 of electrical stimulator 4. The relationship defines a ratio of values for a therapy parameter of one or more electrodes to values of one or more other electrodes 48 used to deliver stimulation. The therapy parameter defines electrical stimulation delivered via the electrode 48 and may include, e.g., one of a current amplitude or a voltage amplitude, an electrical stimulation pulse count, a frequency, etc. External programmer 40 performs a master adjustment to adjust each value of the therapy parameters of each of the multiple electrodes 48 by an amount specified by the relationship to maintain the ratio of the values of the therapy parameters of the multiple electrodes 48. External programmer 40 controls electrical stimulator 4 to deliver electrical stimulation therapy to patient 6 in accordance with the master adjustment. Electrical stimulator 4 may use stimulation generation circuitry 60 to deliver electrical stimulation via each of electrodes 48 that has, e.g., a fractional current amplitude of a current amplitude of each other electrode 48 so as to achieve the master adjustment.

FIG. 5 is a flowchart illustrating an example operation for switching from bipolar stimulation to unipolar stimulation. Specifically, FIG. 5 illustrates an example operation for determining a unipolar electrode combination based on a VNA corresponding to a bipolar electrode combination. For convenience, FIG. 5 is described with respect to programmer 40 and processing circuitry 53 of FIGS. 1 and 2.

In the example of FIG. 5, processing circuitry 53 receives a first stimulation parameter set including a bipolar electrode combination (502). This first stimulation parameter set may be selected by the user or automatically selected by processing circuitry 53 to treat the patient of one or more symptoms. In particular, one or more parameters, including the bipolar electrode combination, may be selected to achieve symptom treatment and/or reduce potential undesired side effects. For example, a clinician may select the placement of anodes and cathodes, and in some examples, amplitudes, pulse widths, and/or frequencies, to generate an electric field that activates desired nerves or neurons.

Processing circuitry 53 can then estimate a VNA that corresponds to the first stimulation parameter set (504). This process may be initiated by a user selecting a selectable icon (e.g., a convert button) associated with converting the bipolar electrode combination to a unipolar electrode combination or an automated process associated with identifying energy efficient electrode combinations or alternatives for therapy. In some examples, processing circuitry 53 may generate the VNA may calculating an electrical field generated by the parameter set and applying the electrical field to a model of tissue that is generic to the patient or patient-specific. Based on this first VNA, processing circuitry 53 may determine a second stimulation parameter set including a unipolar electrode combination (506). In this process, processing circuitry 53 may calculate several VNAs for a plurality of unipolar electrode combinations and compare these VNAs to the first VNA to identify a best fit or otherwise select a similar VNA and associated unipolar electrode combination. In some examples, processing circuitry 53 may back-calculate the parameter set using a unipolar electrode combination that achieves a similar VNA.

In response to determining the second stimulation parameter set that includes the unipolar electrode combination, processing circuitry 53 can control IMD 14 to deliver the second electrical stimulation using the unipolar electrode combination instead of the first electrical stimulation using the bipolar electrode combination (508). In some examples, processing circuitry 53 may enable the user to select an icon that causes processing circuitry 53 to revert back to the prior bipolar electrode combination and parameter set in order to return to the prior stimulation therapy if desired.

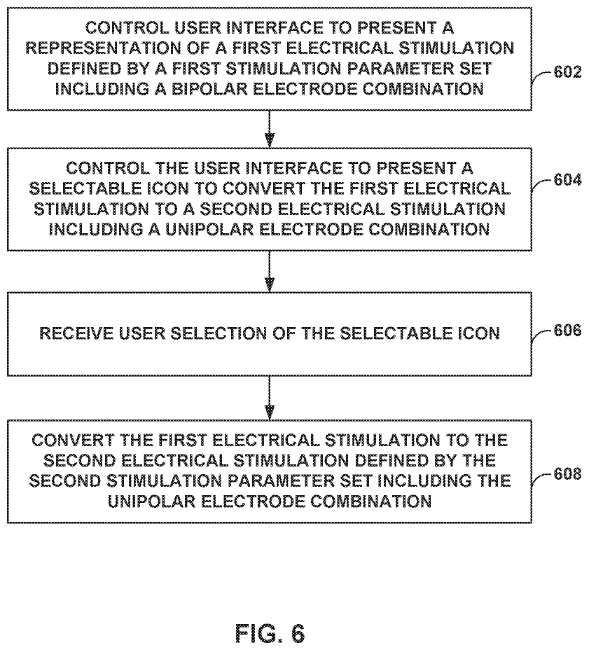

FIG. 6 is a flowchart illustrating an example operation for converting bipolar stimulation to unipolar stimulation in response to user input. Specifically, FIG. 6 illustrates an example operation for determining a unipolar electrode combination from a bipolar electrode combination in response to a user request. For convenience, FIG. 6 is described with respect to programmer 40 and processing circuitry 53 of FIGS. 1 and 2.

In the example of FIG. 6, processing circuitry 53 controls user interface 59 to present a representation of a first electrical stimulation defined by a first stimulation parameter set including a bipolar electrode combination (602). The representation may be a graphical depiction of the VNA corresponding to the first electrical stimulation, one or more numerical representations of the VNA, or even just the first stimulation parameter set. Processing circuitry 53 can also control user interface 59 to present a selectable icon (e.g., a convert button) configured to convert the first electrical stimulation to a second electrical stimulation including a unipolar electrode combination (604). The selectable icon may be displayed before or after the representation of the first electrical stimulation is presented.

In response to receiving user selection of the selectable icon (606), processing circuitry 53 converts the first electrical stimulation to the second electrical stimulation defined by the second stimulation parameter set including the unipolar electrode combination (608). Processing circuitry 53 may convert the electrical stimulation by any technique described herein, such as by comparing candidate VNAs from respective unipolar electrode combinations, switching all anodes to cathodes and adding an anode remote from the leads and/or target tissue, or any combination thereof. Processing circuitry 53 can control IMD 14 to deliver the second electrical stimulation using the unipolar electrode combination instead of the first electrical stimulation using the bipolar electrode combination (508). In some examples, processing circuitry 53 may enable the user to select the selectable icon again which causes processing circuitry 53 to revert back to the prior bipolar electrode combination and parameter set in order to return to the prior stimulation therapy if desired.

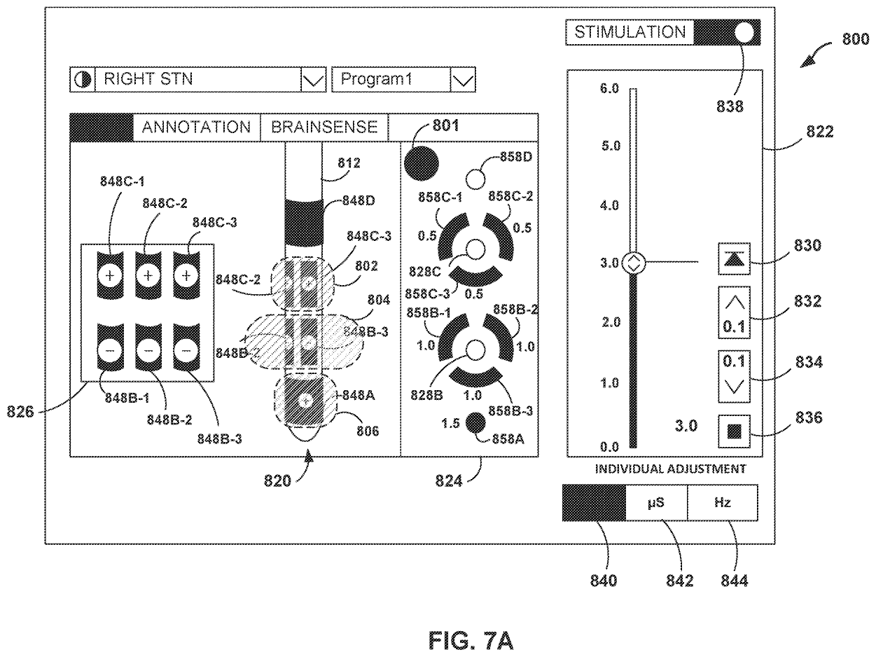

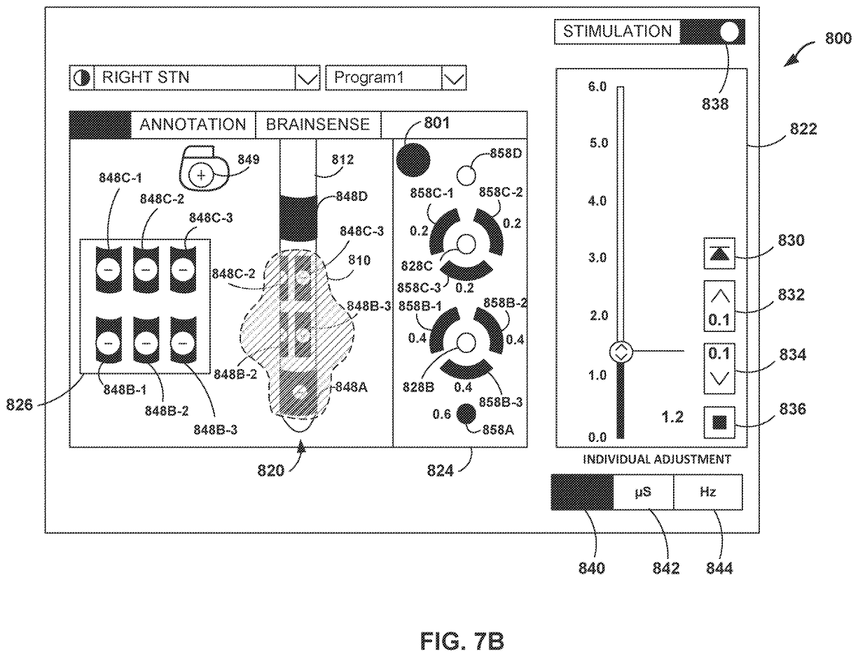

FIGS. 7A and 7B are conceptual diagrams illustrating screens of an example user interface in accordance with the techniques of the disclosure. User interface 800 may be an example of user interface 59 of programmer 40 of FIG. 2. As depicted in the example of FIG. 7A, user interface 800 depicts a representation of electrode icons 848A, 848B-1, 848B-2, 848B-3, 848C-1, 848C-2, 848C-3, and 848D (hereinafter, “electrode icons 848”) disposed on lead 812 within display region 820. In the example of FIGS. 7A and 7B, each of electrode icons 848A-848D correspond to a respective one of electrodes 48 of FIGS. 1 and 3, and lead icon 812 corresponds to lead 12 of FIG. 1.

In the example of FIG. 7A, electrode icons 848B-1, 848B-2, 848B-3 (collectively, “electrode icons 848B”) represent a first subset of electrodes 48 disposed at different circumferential positions around lead 12. Similarly, electrode icons 848C-1, 848C-2, 848C-3 (collectively, “electrode icons 848C”) represent a second subset of electrodes 48 disposed at different circumferential positions around lead 12. While in the example of FIG. 7A, each of the first subset of electrodes 48 and second subset of electrodes 48 are represented by three electrode icons (e.g., electrode icons 848B-1, 848B-2, 848B-3 in the first subset and electrode icons 848C-1, 848C-2, 848C-3 in the second subset), in other examples each subset of electrodes 48 may have any number of electrodes (e.g., more than or fewer than 3 electrodes per subset). Furthermore, while in the example of FIG. 7A, lead icon 812 has two rings of subsets of electrode icons 848, in other examples lead icon 812 may have more rings, fewer rings, or no rings of electrode icons 848, each ring including a subset of one or more electrodes 848, dependent on the actual configuration of lead 12.

In the example of FIG. 7A, electrode icons 848B-1, 848B-2, 848B-3 indicate that corresponding electrodes 48 are selected to act as cathodes for delivery of electrical stimulation by electrical stimulator 4 of FIG. 1. Ring electrode 848A and electrodes 848C-1, 848C-2, 848C-3 indicates that corresponding electrodes 48 are selected to act as anodes. In this configuration of FIG. 7A, the selected electrodes are in a bipolar electrode configuration to generate the VNAs 802, 804, and 806. In some examples, VNAs 802, 804, and 806 may be referenced and/or displayed as a single VNA whether or not there is overlap between portions of the VNAs. Display region 820 further depicts a representation of the VNAs 802, 804, and 806 generated by delivery of electrical stimulation by electrical stimulator 4 according to therapy parameters selected for electrodes corresponding to electrode icons 848B-1, 848B-2, 848B-3, 848C-1, 848C-2, 848C-3, and 848A. Although VNAs 802, 804, and 806 are shows as discrete VNAs in the example of FIG. 7A, they may be referred to collectively as a single VNA in some examples.

User interface 800 includes a toggle button 838 that allows a clinician to activate or deactivate delivery of electrical stimulation by electrical stimulator 4 according to therapy parameters selected for electrodes identified as cathodes and anodes. User interface 800 further includes electrode status window 826, which displays a side view of the status of electrodes 48. For example, as depicted in the example of FIG. 7A, electrode status window 826 depicts electrode icons 848B-1, 848B-2, 848B-3, 848C-1, 848C-2, and 848C-3 indicating corresponding electrodes 48 as acting as anodes for delivery of electrical stimulation by electrical stimulator 4. For implementations where lead 12 includes a subset of electrodes 48 disposed on a ring around lead 12, such as the case for, e.g., electrodes corresponding to electrode icons 848B-1, 848B-2, 848B-3, 848C-1, 848C-2, and 848C-3, the use of electrode status window 826 may assist the clinician in viewing a status of each of electrodes 48 where one or more of the electrode icons 848 may be obscured from view by the 3-dimensional depiction of lead icon 812 within display region 820. For example, as depicted in the example of FIG. 7A, electrode icons 848B-1 and 848C-1 are at least partially obscured by the 3-dimensional depiction of lead icon 812 within display window 820.

User interface 800 further includes electrode selection panel 824. As depicted in FIG. 7A, electrode selection panel 824 includes indicators 858A, 858B-1, 858B-2, 858B-3, 858C-1, 858C-2, 858C-3, and 858D that each depict a selection status for a corresponding axial representation (e.g., a cross-sectional view of the different axial positions corresponding to electrode locations) of one of electrode icons 848A, 848B-1, 848B-2, 848B-3, 848C-1, 848C-2, 848C-3, and 848D. As depicted in FIG. 7A, indicators 858B (shaded in black) denote that the clinician has selected electrodes corresponding to electrode icons 858B for therapy parameter adjustment (e.g., via therapy parameter control panel 822). As depicted in electrode selection panel 824, the clinician has set a value of 3.0 milliamps for a current amplitude of electrical stimulation delivered via each of electrodes corresponding to electrode icons 858B. Indicators 858C and 858A (shaded in gray) denote that the clinician has selected electrodes 48 corresponding to electrode icons 858C and 858A for delivery of stimulation but is not currently adjusting the therapy parameters of such electrodes. As depicted in electrode selection panel 824, the clinician has previously set a value of 0.5 milliamps for a current amplitude of electrical stimulation delivered via each of electrodes 48 corresponding to electrode icons 848C-1, 848C-2, and 848C-3, and a value of 1.5 milliamps for electrode 848A. Indicators 858D (shaded in white) denote that electrodes 48 corresponding to electrode icon 858D are not currently used for delivery of stimulation. The anodes and cathodes indicated as active in the example of FIG. 7A are an example bipolar electrode combination.

In some examples, when active, indicators 858 display a value of an amplitude and a label in both the interactive and selected state. Indicators 858 controls are single-select options, e.g., tapping a second button switches from the first to the second. Each indicator 858 displays an amplitude of a corresponding single electrode 48. In some examples, display window 820 displays a total amplitude for each ring or an amplitude for each electrode within each ring.

Electrode selection panel 824 further includes ring toggle buttons 828B and 828C. Ring toggle buttons 828B and 828C allow a clinician to toggle on or off all of the electrodes of a ring with one button. For example, a clinician may select ring toggle button 828B to transition each of electrodes 48 corresponding to electrode icons 848B-1, 848B-2, and 848B-3 to an “on” state. In some examples, when selecting ring toggle button 828B, each of electrodes corresponding to electrode icons 848B-1, 848B-2, and 848B-3 that were previously in an “off” state may transition to an “on” state and use a predefined value for a therapy parameter (e.g., an initial current amplitude) for delivery of electrical stimulation. Furthermore, a clinician may select ring toggle button 828B a second time to transition each of electrodes corresponding to electrode icons 848B-1, 848B-2, and 848B-3 to an “off” state.

In some examples, each ring toggle button 828B, 828C selects only the available electrodes on the same ring that are active. For example, if only two segments 858C-1 and 858C3 are part of the configuration, ring toggle button 828C only selects 858C-1 and 858C3 and not 858C-2.

User interface 800 further includes therapy parameter control panel 822 that allows a clinician to adjust values of therapy parameters for one or more currently-selected electrodes 48. In the example of FIG. 7A, the clinician has selected electrodes 48 corresponding to electrode icons 848B-1, 848B-2, and 848B-3 and set a value of 1.0 milliamps for a current amplitude of electrical stimulation delivered via the electrodes. Therapy parameter control panel 822 may operate such that the user may adjust the value of the therapy parameter for electrodes 48 corresponding to electrode icons 848B-1, 848B-2, and 848B-3 by pressing incremental increase button 832, incremental decrease button 834, maximum button 830, or minimum button 836. Furthermore, the clinician may select the type of therapy parameter (e.g., current amplitude, pulse duration, or pulse frequency) by selecting a corresponding therapy parameter type button such as milliamp button 840, pulse duration button 842, or pulse frequency button 844.

The status color of indicators 858 (e.g., black, gray, or white) indicates the current status of therapy delivery by electrical stimulator 4 using respective electrodes 48. As an example, where user interface 800 is a touch-sensitive display, a clinician may select a particular electrode 48 for adjustment via therapy parameter control panel 822 by pressing an indicator 858 that corresponds to the desired electrode 48. Further, therapy parameter control panel 822 may automatically update to display values of the therapy parameter for the currently-selected electrode 48. The colors of FIG. 7A are provided for ease of illustration only, and other colors may be used to indicate various statuses or configurations of electrodes 48.

Electrode selection panel 824 includes convert button 801. As described herein, user selection of convert button 801 may cause processing circuitry 53 to convert the bipolar electrode combination of FIG. 7A to a unipolar electrode combination that may have a similar VNA to VNAs 802, 804, and 806. For example, user selection of convert button 801 may cause user interface 800 to change to FIG. 7B.

As shown in the example of FIG. 7B, processing circuitry 53 has converted the bipolar electrode combination of FIG. 7A to the unipolar electrode combination of FIG. 7B. As described herein, processing circuitry 53 may identify a unipolar electrode combination and other parameter values, such as amplitude, that result in a VNA that is similar to the VNA of the previous bipolar electrode combination. In the example of FIG. 7B, this conversion has resulted in changing electrodes 848C-1, 848C-2, 848C-3, and 848A from anodes to cathodes. In addition, the anode has been assigned to electrode 849 which is shown as a representation of the housing of IMD 14.

VNA 810 is a representation of the tissue that is activated by the electrical stimulation delivered by the unipolar electrode combination of FIG. 7B. Each of icons 858 can be selected to adjust the stimulation amplitude. The “black” icons 858B-1, 858B-2, and 858B-3 indicate that these electrodes are selected and can be controlled by pressing incremental increase button 832 or incremental decrease button 834. In this manner, processing circuitry can adjust individual or all electrodes at once as selected by the user. In the example of FIG. 7B, a total of 2.4 mA may be delivered from the cathodes that are selected, which may be less than the current needed in FIG. 7A while still achieving VNA 810 that is similar to the total of VNAs 802, 804, and 806. User selection of convert button 801 another time may cause processing circuitry 53 to revert back to the previous bipolar electrode combination. In some examples, selection of convert button 801 may cause the stimulation to be turned off such that the user can review the unipolar combination before starting stimulation once again. In other examples, stimulation may continue when converting in order to determine if the patient can perceive a difference to the change in stimulation.

The following examples are described herein.
Example 1: A system includes a memory; and processing circuitry operably coupled to the memory and configured to: receive a first stimulation parameter set comprising a bipolar electrode combination that defines a first electrical stimulation; estimate a volume of neural activation (VNA) corresponding to the first stimulation parameter set; determine, based on the first VNA, a second stimulation parameter set comprising a unipolar electrode combination that defines a second electrical stimulation; and control an implantable medical device to deliver the second electrical stimulation instead of the first electrical stimulation.
Example 2: The system of example 1, wherein the processing circuitry is configured to determine the second stimulation parameter set by at least: selecting one or more cathodes on a lead that carried the bipolar electrode combination, the unipolar electrode combination comprising the one or more cathodes and at least one anode remote from the lead; estimating a second VNA corresponding to stimulation generated by the unipolar electrode combination; identifying an amplitude value for the one or more cathodes at which one or more characteristics of the second VNA corresponds to one or more characteristics of the first VNA; and generating the second stimulation parameter set to include the one or more cathodes and the amplitude value.
Example 3: The system of example 2, wherein the amplitude value is a second amplitude value less than a first amplitude value of the first stimulation parameter set.
Example 4: The system of any of examples 2 and 3, the processing circuitry is configured to select the one or more cathodes on the lead by at least keeping any cathodes of the bipolar electrode combination and switching any anodes of the bipolar electrode combination to cathodes.
Example 5: The system of any of examples 2 through 4, wherein the one or more characteristics comprise at least one of a volume, a distance from a respective electrode, or a distance from the lead at a respective axial position, and wherein the processing circuitry is configured to identify the amplitude value for the one or more cathodes at which one or more characteristics of the second VNA corresponds to one or more characteristics of the first VNA.
Example 6: The system of any of examples 2 through 5, wherein the one or more characteristics of the second VNA comprises a plurality of characteristics of the second VNA, wherein the one or more characteristics of the first VNA comprises a plurality of characteristics of the first VNA, and wherein different characteristics identifying the amplitude value for the one or more cathodes based on differently weighted characteristics of the one or more characteristics of the first VNA.
Example 7: The system of any of examples 1 through 6, wherein the bipolar electrode combination comprises at least one anode disposed on an implantable medical lead and at least one cathode disposed on the implantable medical lead.
Example 8: The system of any of examples 1 through 7, wherein the unipolar electrode combination comprises at least one anode disposed on a housing of the implantable medical device and at least one cathode disposed on an implantable medical lead coupled to the implantable medical device.
Example 9: The system of any of examples 1 through 8, wherein the second stimulation parameter set comprises at least one of an amplitude, a pulse width, and a pulse frequency.
Example 10: The system of any of examples 1 through 9, wherein the bipolar electrode combination comprises at least one electrode of a plurality of electrodes disposed at different locations around a perimeter of a lead.
Example 11: The system of any of examples 1 through 10, further comprising a lead comprising a plurality of electrodes from which a subset of electrodes correspond to the bipolar electrode combination.
Example 12: The system of any of examples 1 through 11, further comprising an external programmer that comprises the processing circuitry and the memory.
Example 13: The system of any of examples 1 through 12, wherein the system further comprises a display, and wherein the processing circuitry is further configured to control the display to present a selectable icon that, when selected, causes the processing circuitry to determine, based on the first VNA, the second stimulation parameter set comprising the unipolar electrode combination that defines the second electrical stimulation.
Example 14: A method includes receiving, by processing circuitry, a first stimulation parameter set comprising a bipolar electrode combination that defines a first electrical stimulation; estimating, by the processing circuitry, a volume of neural activation (VNA) corresponding to the first stimulation parameter set; determining, by the processing circuitry and based on the first VNA, a second stimulation parameter set comprising a unipolar electrode combination that defines a second electrical stimulation; and controlling, by the processing circuitry, an implantable medical device to deliver the second electrical stimulation instead of the first electrical stimulation.
Example 15: The method of example 14, wherein determining the second stimulation parameter set comprises: selecting one or more cathodes on a lead that carried the bipolar electrode combination, the unipolar electrode combination comprising the one or more cathodes and at least one anode remote from the lead; estimating a second VNA corresponding to stimulation generated by the unipolar electrode combination, identifying an amplitude value for the one or more cathodes at which one or more characteristics of the second VNA corresponds to one or more characteristics of the first VNA; and generating the second stimulation parameter set to include the one or more cathodes and the amplitude value.
Example 16: The method of example 15, wherein the amplitude value is a second amplitude value less than a first amplitude value of the first stimulation parameter set.
Example 17: The method of any of examples 15 and 16, wherein selecting the one or more cathodes on the lead comprises keeping any cathodes of the bipolar electrode combination and switching any anodes of the bipolar electrode combination to cathodes.
Example 18: The method of any of examples 15 through 17, wherein the one or more characteristics comprise at least one of a volume, a distance from a respective electrode, or a distance from the lead at a respective axial position, and further comprising identifying the amplitude value for the one or more cathodes at which one or more characteristics of the second VNA corresponds to one or more characteristics of the first VNA.
Example 19: The method of any of examples 15 through 18, wherein the one or more characteristics of the second VNA comprises a plurality of characteristics of the second VNA, wherein the one or more characteristics of the first VNA comprises a plurality of characteristics of the first VNA, and wherein different characteristics identifying the amplitude value for the one or more cathodes based on differently weighted characteristics of the one or more characteristics of the first VNA.
Example 20: The method of any of examples 14 through 19, wherein the bipolar electrode combination comprises at least one anode disposed on an implantable medical lead and at least one cathode disposed on the implantable medical lead.
Example 21: The method of any of examples 14 through 20, wherein the unipolar electrode combination comprises at least one anode disposed on a housing of the implantable medical device and at least one cathode disposed on an implantable medical lead coupled to the implantable medical device.
Example 22: The method of any of examples 14 through 21, wherein the second stimulation parameter set comprises at least one of an amplitude, a pulse width, and a pulse frequency.
Example 23: The method of any of examples 14 through 22, wherein the bipolar electrode combination comprises at least one electrode of a plurality of electrodes disposed at different locations around a perimeter of a lead.
Example 24: The method of any of examples 14 through 23, further comprising controlling a display to present a selectable icon that, when selected, causes the processing circuitry to determine, based on the first VNA, the second stimulation parameter set comprising the unipolar electrode combination that defines the second electrical stimulation.
Example 25: A system includes a memory including a first stimulation parameter set that defines a first electrical stimulation, and processing circuitry configured to control a user interface to present a representation of first electrical stimulation defined by a first stimulation parameter set comprising a bipolar electrode combination; controlling, by the processing circuitry, the user interface to present a selectable icon associated with converting the first electrical stimulation defined by the first stimulation parameter set comprising the bipolar electrode combination to a second electrical stimulation defined by a second stimulation parameter set comprising a unipolar electrode combination; receiving, by the processing circuitry, user selection of the selectable icon; and responsive to receiving the user selection of the selectable icon, converting the first electrical stimulation to the second electrical stimulation defined by the second stimulation parameter set comprising the unipolar electrode combination.
Example 26: The system of example 25, wherein the processor is configured to convert the first electrical stimulation to the second electrical stimulation by at least comparing a second volume of neural activation (VNA) associated with the second electrical stimulation to a first VNA associated with the first electrical stimulation.
Example 27: The system of any of examples 25 and 26, wherein the representation comprises a visual representation of the VNA, and wherein the processor is configured to control the user interface to present a visual representation of the VNA corresponding to the second electrical stimulation.
Example 28: The system of any of examples 25 through 27, wherein the processing circuitry is configured to control the user interface to present one or more controls configured to receive user input adjusting a value of respective stimulation parameters of the second stimulation parameter set.
Example 29: The system of any of examples 25 through 28, wherein the processing circuitry is configured to toggle between the first electrical stimulation comprising the bipolar electrode combination and the second electrical stimulation comprising the unipolar electrode combination in response to user selection of the selectable icon.
Example 30: The system of any of examples 25 through 29, further comprising a display configured to present the user interface that comprises the selectable icon.
Example 31: The system of any of examples 26 through 30, further comprising an external programmer comprising the processing circuitry and the display.
Example 32: The system of any of examples 25 through 31, further comprising an implantable medical device configured to deliver the first electrical stimulation and the second electrical stimulation.
Example 33: A method includes controlling, by processing circuitry, a user interface to present a representation of first electrical stimulation defined by a first stimulation parameter set comprising a bipolar electrode combination; controlling, by the processing circuitry, the user interface to present a selectable icon associated with converting the first electrical stimulation defined by the first stimulation parameter set comprising the bipolar electrode combination to a second electrical stimulation defined by a second stimulation parameter set comprising a unipolar electrode combination; receiving, by the processing circuitry, user selection of the selectable icon; and responsive to receiving the user selection of the selectable icon, converting the first electrical stimulation to the second electrical stimulation defined by the second stimulation parameter set comprising the unipolar electrode combination.
Example 34: The method of example 33, wherein converting the first electrical stimulation to the second electrical stimulation comprises comparing a second volume of neural activation (VNA) associated with the second electrical stimulation to a first VNA associated with the first electrical stimulation.
Example 35: The method of any of examples 33 and 34, wherein the representation comprises a visual representation of the VNA, and wherein the method further comprises controlling the user interface to present a visual representation of the VNA corresponding to the second electrical stimulation.
Example 36: The method of any of examples 33 through 35, further comprising controlling the user interface to present one or more controls configured to receive user input adjusting a value of respective stimulation parameters of the second stimulation parameter set.
Example 37: The method of any of examples 33 through 36, further comprising toggling between the first electrical stimulation comprising the bipolar electrode combination and the second electrical stimulation comprising the unipolar electrode combination in response to user selection of the selectable icon.
Example 38: The method of any of examples 33 through 37, further comprising presenting, by a display the user interface that comprises the selectable icon.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system comprising:
a memory; and
processing circuitry operably coupled to the memory and configured to:
receive a first stimulation parameter set comprising a bipolar electrode combination that defines a first electrical stimulation;
estimate a volume of neural activation (VNA) corresponding to the first stimulation parameter set;
determine, based on the first VNA, a second stimulation parameter set comprising a unipolar electrode combination that defines a second electrical stimulation; and
control an implantable medical device to deliver the second electrical stimulation instead of the first electrical stimulation.

2. The system of claim 1, wherein the processing circuitry is configured to determine the second stimulation parameter set by at least:
selecting one or more cathodes on a lead that carried the bipolar electrode combination, the unipolar electrode combination comprising the one or more cathodes and at least one anode remote from the lead;
estimating a second VNA corresponding to stimulation generated by the unipolar electrode combination;
identifying an amplitude value for the one or more cathodes at which one or more characteristics of the second VNA corresponds to one or more characteristics of the first VNA; and
generating the second stimulation parameter set to include the one or more cathodes and the amplitude value.

3. The system of claim 2, wherein the amplitude value is a second amplitude value less than a first amplitude value of the first stimulation parameter set.

4. The system of claim 2, the processing circuitry is configured to select the one or more cathodes on the lead by at least keeping any cathodes of the bipolar electrode combination and switching any anodes of the bipolar electrode combination to cathodes.

5. The system of claim 2, wherein the one or more characteristics comprise at least one of a volume, a distance from a respective electrode, or a distance from the lead at a respective axial position, and wherein the processing circuitry is configured to identify the amplitude value for the one or more cathodes at which one or more characteristics of the second VNA corresponds to one or more characteristics of the first VNA.

6. The system of claim 2, wherein the one or more characteristics of the second VNA comprises a plurality of characteristics of the second VNA, wherein the one or more characteristics of the first VNA comprises a plurality of characteristics of the first VNA, and wherein different characteristics identifying the amplitude value for the one or more cathodes based on differently weighted characteristics of the one or more characteristics of the first VNA.

7. The system of claim 1, wherein the bipolar electrode combination comprises at least one anode disposed on an implantable medical lead and at least one cathode disposed on the implantable medical lead.

8. The system of claim 1, wherein the unipolar electrode combination comprises at least one anode disposed on a housing of the implantable medical device and at least one cathode disposed on an implantable medical lead coupled to the implantable medical device.

9. The system of claim 1, wherein the second stimulation parameter set comprises at least one of an amplitude, a pulse width, and a pulse frequency.

10. The system of claim 1, wherein the bipolar electrode combination comprises at least one electrode of a plurality of electrodes disposed at different locations around a perimeter of a lead.

11. The system of claim 1,
wherein the system further comprises a display, and
wherein the processing circuitry is further configured to control the display to present a selectable icon that, when selected, causes the processing circuitry to determine, based on the first VNA, the second stimulation parameter set comprising the unipolar electrode combination that defines the second electrical stimulation.

12. A method comprising:
receiving, by processing circuitry, a first stimulation parameter set comprising a bipolar electrode combination that defines a first electrical stimulation;
estimating, by the processing circuitry, a volume of neural activation (VNA) corresponding to the first stimulation parameter set;
determining, by the processing circuitry and based on the first VNA, a second stimulation parameter set comprising a unipolar electrode combination that defines a second electrical stimulation; and
controlling, by the processing circuitry, an implantable medical device to deliver the second electrical stimulation instead of the first electrical stimulation.

13. The method of claim 12, wherein determining the second stimulation parameter set comprises:
selecting one or more cathodes on a lead that carried the bipolar electrode combination, the unipolar electrode combination comprising the one or more cathodes and at least one anode remote from the lead;
estimating a second VNA corresponding to stimulation generated by the unipolar electrode combination;
identifying an amplitude value for the one or more cathodes at which one or more characteristics of the second VNA corresponds to one or more characteristics of the first VNA; and
generating the second stimulation parameter set to include the one or more cathodes and the amplitude value.

14. The method of claim 13, wherein the amplitude value is a second amplitude value less than a first amplitude value of the first stimulation parameter set.

15. The method of claim 14, wherein selecting the one or more cathodes on the lead comprises keeping any cathodes of the bipolar electrode combination and switching any anodes of the bipolar electrode combination to cathodes.

16. The method of claim 13, wherein the one or more characteristics comprise at least one of a volume, a distance from a respective electrode, or a distance from the lead at a respective axial position, and further comprising identifying the amplitude value for the one or more cathodes at which one or more characteristics of the second VNA corresponds to one or more characteristics of the first VNA.

17. The method of claim 13, wherein the one or more characteristics of the second VNA comprises a plurality of characteristics of the second VNA, wherein the one or more characteristics of the first VNA comprises a plurality of characteristics of the first VNA, and wherein different characteristics identifying the amplitude value for the one or more cathodes based on differently weighted characteristics of the one or more characteristics of the first VNA.

18. The method of claim 12, wherein the unipolar electrode combination comprises at least one anode disposed on a housing of the implantable medical device and at least one cathode disposed on an implantable medical lead coupled to the implantable medical device.

19. The method of claim 12, further comprising controlling a display to present a selectable icon that, when selected, causes the processing circuitry to determine, based on the first VNA, the second stimulation parameter set comprising the unipolar electrode combination that defines the second electrical stimulation.

20. A system comprising:
a memory comprising a first stimulation parameter set that defines a first electrical stimulation; and
processing circuitry operably coupled to the memory and configured to:
control a user interface to present a representation of first electrical stimulation defined by a first stimulation parameter set comprising a bipolar electrode combination;
controlling, by the processing circuitry, the user interface to present a selectable icon associated with converting the first electrical stimulation defined by the first stimulation parameter set comprising the bipolar electrode combination to a second electrical stimulation defined by a second stimulation parameter set comprising a unipolar electrode combination;
receiving, by the processing circuitry, user selection of the selectable icon; and
responsive to receiving the user selection of the selectable icon, converting the first electrical stimulation to the second electrical stimulation defined by the second stimulation parameter set comprising the unipolar electrode combination.
